(19) United States
(12) Patent Application Publication
(10) Pub. No.: US 2017/0281599 A1
(43) Pub. Date: Oct. 5, 2017

(54) AMORPHOUS FORM OF DACLATASVIR AND ITS SALTS AND PROCESS FOR PREPARATION THEREOF

(71) Applicant: CADILA HEALTHCARE LIMITED, Ahmedabad (IN)

(72) Inventors: Sanjay Jagdish DESAI, Ahmedabad - Gujarat (IN); Sandipan Prabhurao BONDGE, Ahmedabad - Gujarat (IN); Naitik Bharatbhai PATEL, Ahmedabad - Gujarat (IN); Chirag Vasantbhai PATEL, Ahmedabad - Gujarat (IN); Sandip Kiritkumar UPADHYAY, Ahmedabad - Gujarat (IN)

(73) Assignee: CADILA HEALTHCARE LIMITED, Ahmedabad (IN)

(21) Appl. No.: 15/471,087

(22) Filed: Mar. 28, 2017

(30) Foreign Application Priority Data
Mar. 29, 2016 (IN) 201621010711

(51) Int. Cl. A61K 31/4178; C07D 403/14; G01N 33/15; B65D 81/26; B65B 7/02; B65D 65/40; B65D 77/04; A61K 9/16; B65B 31/00

(52) U.S. Cl. CPC A61K 31/4178; A61K 9/1635; C07D 403/14; A61K 9/1682; G01N 33/15; B65B 31/00; B65B 7/02; B65D 65/40; B65D 77/04; B65D 81/266

(57) ABSTRACT

This relates to an amorphous form of daclatasvir and its salts and process for preparation thereof. In particular, it relates to an amorphous form of daclatasvir and daclatasvir dihydrochloride. It also relates to pharmaceutical compositions comprising an amorphous form of daclatasvir or daclatasvir dihydrochloride for oral administration for treatment of Hepatitis C virus (HCV).

### FIELD OF THE INVENTION

The field of the invention relates to an amorphous form of daclatasvir and its salts and process for preparation thereof. In particular, the invention relates to an amorphous form of daclatasvir and daclatasvir dihydrochloride. The invention also relates to pharmaceutical compositions comprising an amorphous form of daclatasvir or daclatasvir dihydrochloride for oral administration for treatment of Hepatitis C virus (HCV).

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

The compound methyl ((1S)-1-(((2S)-2-(5-(4′-(2-((2S)-1-((2S)-2-((methoxycarbonyl)amino)-3-methylbutanoyl)-2-pyrrolidinyl)-1H-imidazol-5-yl)-4-biphenylyl)-1H-imidazol-2-yl)-1-pyrrolidinyl)carbonyl)-2-methylpropyl)carbamate dihydrochloride salt represented by Formula (I),

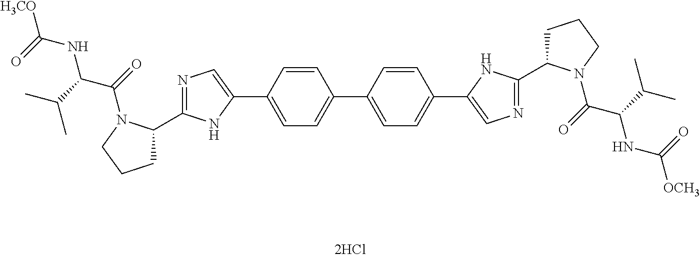 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]

is useful for the treatment of HCV infection.

U.S. Pat. No. 8,329,159 B2 discloses daclatasvir or a pharmaceutically acceptable salt and process for the preparation thereof.

U.S. Pat. Nos. 7,728,027 B2 and 9,006,455 B2 disclose process for preparation of daclatasvir and its pharmaceutically acceptable salt.

U.S. Pat. No. 8,629,171 B2 discloses crystalline Form N-2 of daclatasvir dihydrochloride and process for preparing thereof.

Crystalline solids normally require a significant amount of energy for dissolution due to their highly organized, lattice like structures. For example, the energy required for a drug molecule to escape from a crystal is more than from an amorphous or a non-crystalline form. It is known that the amorphous forms in a number of drugs exhibit different dissolution characteristics and in some cases different bioavailability patterns compared to the crystalline form (Econno T., Chem. Pharm. Bull., 1990; 38: 2003-2007). An amorphous form of drug substances like rosuvastatin calcium, rabeprazole sodium are some of the examples of an amorphous drug exhibiting much higher bioavailability than their crystalline forms, which leads to the selection of the amorphous form as the final drug substance for pharmaceutical dosage form development. Therefore, it is desirable to have an amorphous form of drug substance with high purity, to meet the needs of regulatory agencies and also reproducible processes for their preparation.

The present invention herein provides a stable amorphous form of daclatasvir dihydrochloride. Moreover, the present invention provides a solid amorphous dispersion of daclatasvir dihydrochloride which may be reproduced easily and is amenable for processing into a dosage form.

In view of the above, it is therefore, desirable to provide an efficient, more economical, less hazardous and eco-friendly process for the preparation of an amorphous form of daclatasvir dihydrochloride. The amorphous form provided herein is stable under ordinary stability conditions with respect to purity, storage and is free flowing powder.

### SUMMARY OF THE INVENTION

In one general aspect, there is provided an amorphous solid dispersion comprising daclatasvir dihydrochloride and a polymer.

In another general aspect, there is provided an amorphous form of daclatasvir dihydrochloride.

In another general aspect, there is provided an amorphous form of daclatasvir dihydrochloride having water content from about 0.5% to about 5% wt/wt.

In another general aspect, there is provided a process for preparation of an amorphous form of daclatasvir dihydrochloride, the process comprising:
(a) providing a solution of daclatasvir dihydrochloride in one or more solvents; and
(b) obtaining an amorphous form of daclatasvir dihydrochloride by removal of the solvent.

In another general aspect, there is provided a stable amorphous form of daclatasvir dihydrochloride, which is at least stable during storage and drying.

In another general aspect, the amorphous form of daclatasvir dihydrochloride is stabilized by placing the amorphous daclatasvir dihydrochloride under nitrogen atmosphere in a double polythene bag and sealing it to obtain primary packing; placing the primary packing inside a double polyethylene bag optionally containing oxygen busters and sealing it to obtain secondary packing; placing the secondary packing inside a triple laminated bag optionally containing oxygen busters and sealing it; and placing the sealed triple laminated bag inside a high density polyethylene (HDPE) container and storing in controlled environment at a temperature of about 25° C. to 40° C. at a relative humidity from 60% RH to about 75% RH, respectively.

In another general aspect, there is provided an amorphous daclatasvir dihydrochloride having particle size distributions wherein the 10th volume percentile particle size (D10) is about 50 μm or less, the 50th volume percentile particle size (D50) is about 200 μm or less, and the 90th volume percentile particle size (D90) is about 400 μm or less, or any combination thereof.

In another general aspect, there is provided a process for the preparation of amorphous form of daclatasvir dihydrochloride, the process comprising:
(a) suspending daclatasvir dihydrochloride in one or more solvents;
(b) removing the solvent; and
(c) obtaining the amorphous form of daclatasvir dihydrochloride.

In another general aspect, there is provided a composition comprising an amorphous form of daclatasvir dihydrochloride and a polymer, as well as processes for preparing compositions from a solvent-based medium.

In another general aspect, there is provided an amorphous form of daclatasvir dihydro chloride having purity of about 95% or more, of about 98% or more, of about 99% or more, of about 99.5% or more, of about 99.8% or more, of about 99.9% or more, as determined using high performance liquid chromatography (HPLC).

In another general aspect, there is provided a pharmaceutical composition comprising an amorphous form of daclatasvir dihydrochloride having one or more pharmaceutically acceptable carriers, excipients or diluents.

In another general aspect, there is provided a pharmaceutical composition comprising a storage stable amorphous form of daclatasvir dihydrochloride having one or more pharmaceutically acceptable carriers, excipients or diluents.

### BRIEF DESCRIPTION OF THE ACCOMPANYING DRAWINGS

FIG. 1. Shows the X-ray diffractogram (XRD) of the amorphous solid dispersion of daclatasvir dihydrochloride with Povidone (K-30 USP) as per Example-1.

FIG. 2. Shows the X-ray diffractogram (XRD) of the amorphous form of daclatasvir dihydrochloride as per Example-2.

FIG. 3. Shows the X-ray diffractogram (XRD) of the amorphous form of daclatasvir dihydrochloride as per Example-3.

FIG. 4. Shows the X-ray diffractogram (XRD) of the amorphous form of daclatasvir dihydrochloride as per Example-4.

FIG. 5. Shows the X-ray diffractogram (XRD) of the amorphous form of daclatasvir dihydrochloride as per Example-5.

### DETAILED DESCRIPTION OF THE INVENTION

The above and other objects of the present invention are achieved by the process of the present invention, which leads to greater stability of amorphous form of daclatasvir dihydrochloride. The invention provides a process for preparing amorphous form of daclatasvir dihydrochloride in an organic solvent.

Optionally, the solution, prior to any solids formation, can be filtered to remove any undissolved solids, solid impurities and the like prior to removal of the solvent. Any filtration system and filtration techniques known in the art can be used.

As used herein, the terms “suspending”, “slurrying” and “triturating” are interchange able, and refer to a process carried out in a heterogeneous mixture where complete dissolution does not occur. Also, heating the suspension or slurry can result in a homogenous mixture where complete or partial dissolution occurs at an elevated temperature or ambient temperature.

All ranges recited herein include the endpoints, including those that recite a range “between” two values. Terms such as “about”, “generally”. “substantially,” and the like are to be construed as modifying a term or value such that it is not an absolute. Such terms will be defined by the circumstances and the terms that they modify as those terms are understood by those skill in the art. This includes, at very least, the degree of expected experimental error, technique error and instrument error for a given technique used to measure a value.

As used herein, the term “stable daclatasvir dihydrochloride” includes an amorphous form of daclatasvir dihydrochloride that doesn't convert to a crystalline form of daclatasvir dihydrochloride and free from residual solvents, after the amorphous form of daclatasvir dihydrochloride is exposed to a relative humidity of 75% at 40° C. or 60% at 25° C. for a period of at least three months.

As used herein, the term “solid dispersion” means any solid composition having at least two components. In certain embodiments, a solid dispersion as disclosed herein includes an active ingredient daclatasvir dihydrochloride dispersed among at least one other component, for example a polymer.

The term “immobilize” as used herein with reference to the immobilization of the active compound i.e. daclatasvir dihydrochloride in the polymer matrix, means that molecules of the active compound interact with molecules of the polymer in such a way that the molecules of the daclatasvir dihydrochloride are held in the aforementioned matrix and prevented from crystal nucleation due to lack of mobility.

In one general aspect, there is provided an amorphous solid dispersion comprising daclatasvir dihydrochloride and a polymer.

In general, the polymer is selected from an ionic polymer or non-ionic polymer comprising hydroxypropylmethyl cellulose acetate succinate (HPMC-AS), hydroxypropylmethyl cellulose (HPMC), methacrylic acid copolymers, polyvinylpyrrolidone (PVP). In particular, PVP of different grades like K-15, K-30, K-60, K-90 and K-120 may be used for the preparation of amorphous composition. More particularly, HPMC-AS, HPMC or PVP K-30 may be used.

In some embodiments, the daclatasvir dihydrochloride may be dispersed within a matrix formed by a polymer in its solid state such that it is immobilized in its amorphous form. The polymer may prevent intramolecular hydrogen bonding or weak dispersion forces between two or more drug molecules of daclatasvir dihydrochloride. The solid dispersion provides for a large surface area, thus further allowing for improved dissolution and bioavailability of daclatasvir dihydrochloride.

In some embodiments, the ratio of the amount of weight of daclatasvir dihydrochloride within the solid dispersion to the amount by weight of the polymer therein is from about 1:1 to about 1:10. The composition of daclatasvir dihydrochloride with polymer, preferably PVP K-30 or HPMC-AC or HPMC may be prepared by using about 1:1 to about 1:10 polymers with respect to daclatasvir dihydrochloride. The usage of higher molar amount of polymer increases the amorphous character of the drug substance.

In another general aspect, there is provide a process for the preparation of an amorphous solid dispersion comprising daclatasvir dihydrochloride and a polymer, the process comprising mixing daclatasvir dihydrochloride with a polymer in one or more solvents and obtaining the amorphous solid dispersion daclatasvir dihydrochloride by removal of the solvent.

In general, the daclatasvir dihydrochloride and a polymer are dissolved in one or more solvents having a low boiling point, e.g. methanol, ethanol, isopropanol, acetone, and ethyl acetate. The amorphous solid dispersion may be obtained by removal of the solvent (for example by spray drying, lyophilization, flash evaporation, vacuum distillation and the like) thereby leaving the amorphous solid dispersion precipitated in a matrix formed by the polymer.

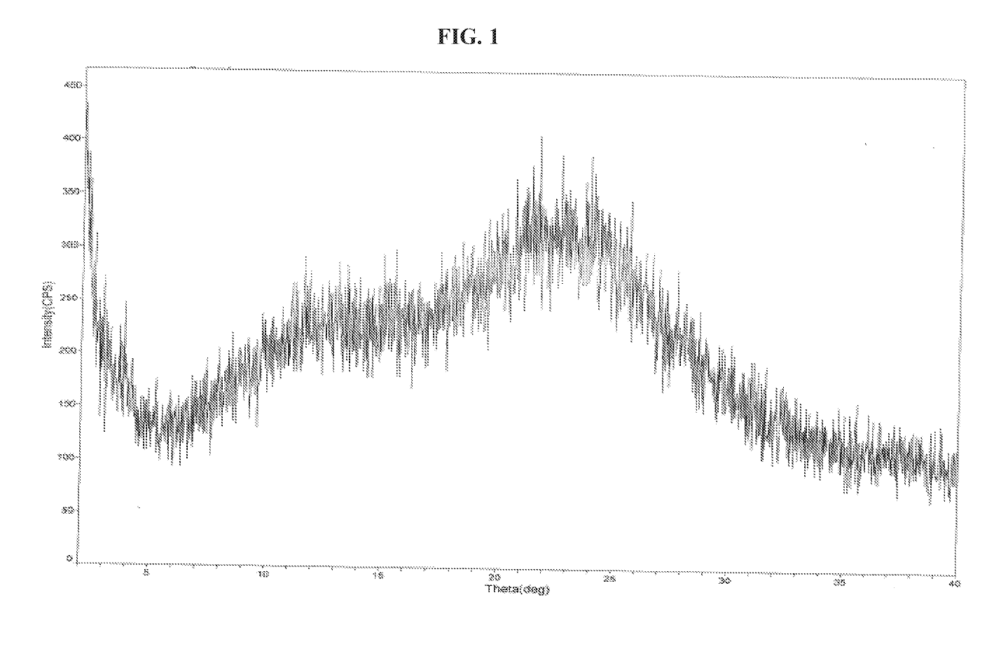

In general, the amorphous solid dispersion of daclatasvir dihydrochloride and a polymer is characterized by XRD pattern substantially as same as that depicted in FIG. 1.

In another general aspect, there is provided an amorphous form of daclatasvir dihydrochloride.

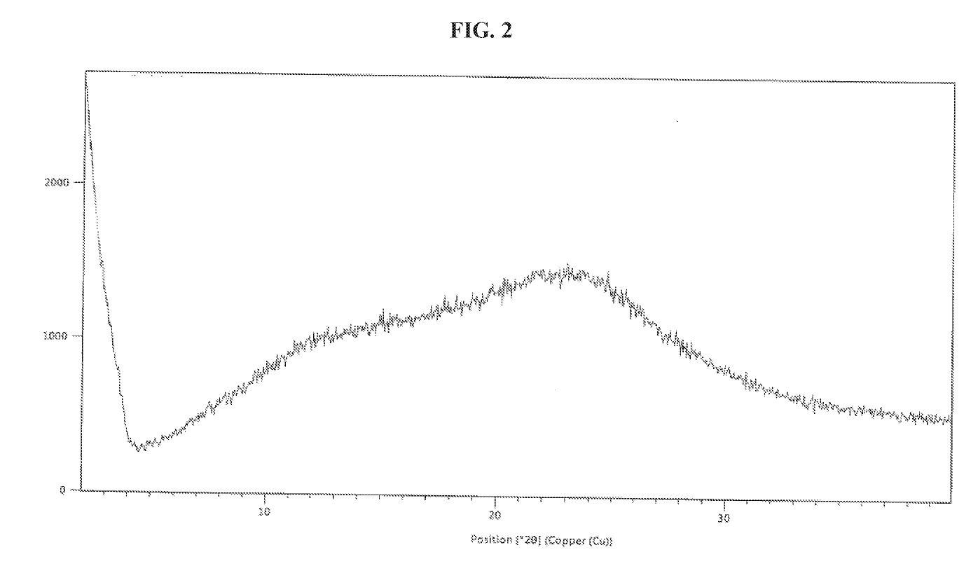

In general, the amorphous form of daclatasvir dihydrochloride is characterized by XRD pattern substantially as same as that depicted in FIG. 2 to FIG. 5.

In another general aspect, there is provided an amorphous form of daclatasvir dihydrochloride having water content from about 0.5% to about 5% wt/wt.

In another general aspect, there is provided an amorphous form of daclatasvir dihydrochloride substantially free from of residual solvents.

In general, the term “free from residual solvents” herein means residual solvents are within the permissible ICH limits suitable for pharmaceutical preparations. For example but not limited to less than 0.5%, particularly less than 0.3% or more particularly less than 0.2%, or most particularly not in detectable amount.

In another general embodiment, there is provided a process for preparation an amorphous form of daclatasvir dihydrochloride, the process comprising:
(a) providing a solution of daclatasvir dihydrochloride in one or more solvents; and
(b) obtaining an amorphous form of daclatasvir dihydrochloride by removal of the solvent.

In general, step (a) involves providing a solution of daclatasvir or a salt thereof in one or more solvent or mixture thereof.

The solution for step (a) can be obtained by the known methods that include:
(i) direct use of a reaction mixture containing daclatasvir dihydrochloride that is obtained in the course of its synthesis; or
(ii) dissolving daclatasvir dihydrochloride in one or more solvent or mixture thereof.

The solvents steps (a) comprises one or more of alcohols selected from methanol, ethanol, isopropanol, 2-propanol, 1-butanol, and t-butyl alcohol; ketones selected from acetone, butanone, and methyl isobutyl ketone; esters selected from ethyl acetate, isopropyl acetate, t-butyl acetate, and isobutyl acetate; halogenated hydrocarbons selected from methylene dichloride, ethylene dichloride, and chlorobenzene; ethers selected from diethyl ether, dimethyl ether, diisopropyl ether and 1,4-dioxane; nitriles selected from acetonitrile, and propionitrile; and polar aprotic solvents selected from N,N-dimethylformamide, N,N-dimethylacetamide. N-methylpyrrolidone, dimethyl sulfoxide, and mixtures thereof.

In general, the step b) involves isolation of an amorphous form of daclatasvir dihydrochloride from the solvents of step (a). The isolation may be affected by removing the solvent. The solvent may be removed by the known techniques in the art but not limited to a rotational distillation device such as a Buchi Rotavapor, spray drying, agitated thin film drying (“ATFD”), and freeze drying (lyophilization).

Alternatively, the isolation can be effected by addition of an anti-solvent to the solution obtain in step a), optionally by concentrating the solution obtained in step a). In general, the anti-solvents comprises one or more of hydrocarbons selected from hexanes, n-heptane, n-pentane, cyclohexane, and methylcyclohexane; aromatic hydrocarbons selected from toluene, xylene, and ethylbenzene; ethers selected from diethyl ether, diisopropyl ether, t-butyl methyl ether, dibutyl ether, tetrahydrofuran, 1,4-dioxane, and 2-methoxyethanol, and water or mixture thereof.

In another general aspect, there is provided spray drying a solution of daclatasvir dihydrochloride that involves the spray drying of feed stock, which is prepared as discussed below, wherein any crystalline form of daclatasvir dihydrochloride may be used. The feedstock is dozed into the spray-drying instrument Labultima Spray Dryer Model LU-222 Advanced model and spray drying is carried out under the following parameters.

Spray Dryer Specification,

| | |
|---|---|
| Labultima Spray Dryer Model | LU-222 Advanced model (twin cyclone) |
| Spray Nozzle | SS316 with 2′O′ rings-0.7 mm. |
| Evaporation rate | 1000 ml of water evaporation/hr at max. |
| | drying air flow rate in insulated condition. |

Parameters of the spry dryer:

| Inlet temperature (° C.) | 75 | |
|---|---|---|
| Outlet temperature (° C.) | 70 | |
| Product temperature (° C.) | 65 | |
| Inlet high temperature (° C.) | 120 | |
| Outlet high temperature (° C.) | 100 | |
| Cooling temperature (° C.) | 50 | |
| Aspirator (Nm3/hr.) | 100 | |
| Feed pump (ml/min) | 3.0 | |
| D-block on time | 1 | Sec |
| D-block off time | 60 | sec |

| | |
|---|---|
| Cycle time (min.) | 999 |
| Air pressure (Kg/cm2) | 0.81 |

In general, the feed stock of daclatasvir dihydrochloride is conveniently prepared by dissolving any known forms or wet cake of daclatasvir dihydrochloride in the solvent selected from one or more of acetone, methanol, ethanol, acetone, ethyl acetate, methylene dichloride, water-methanol or water-ethanol, water-acetone are solvent used or such solvents that evaporate easily to afford dry product, most particularly acetone, methanol, ethanol, ethyl acetate or mixtures thereof.

In another general aspect, there is provided a stable daclatasvir dihydrochloride, which is at least stable during storage and drying.

In another general aspect, the amorphous form of daclatasvir dihydrochloride is stabilized by placing the amorphous daclatasvir dihydrochloride under nitrogen atmosphere in a double polythene bag and sealing it to obtain primary packing; placing the primary packing inside a double polyethylene bag optionally containing oxygen busters and sealing it to obtain secondary packing; placing the secondary packing inside a triple laminated bag optionally containing oxygen busters and sealing it; and placing the sealed triple laminated bag inside a high density polyethylene (HDPE) container and storing in controlled environment at a temperature of about 25° C. to 40° C. at a relative humidity from 60% RH to about 75% RH, respectively.

In general, there is provided a process for stabilizing amorphous form of daclatasvir dihydrochloride by placing the amorphous daclatasvir dihydrochloride under nitrogen atmosphere in a double polythene bag and tying with thread to obtain primary packing; placing the primary packing inside a black colour double polyethylene bag optionally containing oxygen busters and sealing it to obtain secondary packing; placing the secondary packing inside a triple laminated bag optionally containing oxygen busters and sealing it; and placing the scaled triple laminated bag inside a high density polyethylene (HDPE) container and storing in controlled environment at a temperature of about 25° C. to 40° C. at a relative humidity from 60% RH to about 75% RH, respectively.

In general, the stable daclatasvir dihydrochloride includes an amorphous form of daclatasvir dihydrochloride that doesn't convert to a crystalline form of daclatasvir dihydrochloride and free from residual solvents, after the amorphous form of daclatasvir dihydrochloride is exposed to a relative humidity of 75% at 40° C. or 60% at 25° C. for a period of at least three months.

The invention provides stable amorphous form of daclatasvir dihydrochloride having water content from about 0.5% to about 5% wt/wt, which is free flowing and does not develop crystallinity under normal stability conditions for at least about 3 months.

In another general aspect, any form of daclatasvir dihydrochloride can be spray dried by dissolving or suspending or slurring in solvent or solvent-water system to obtain the amorphous form.

In the present invention feedstock of daclatasvir dihydrochloride in solvent or aqueous solvent system is spray-dried. Thus obtain spray-dried compound is in amorphous form, this fact is again confirmed by the X-ray powder diffractogram of spray-dried daclatasvir dihydrochloride.

In another general aspect, there is provided an amorphous form of daclatasvir dihydro chloride having chiral purity of about 95% or more, of about 98% or more, of about 99% or more, of about 99.5% or more, of about 99.8% or more, of about 99.9% or more, as determined using high performance liquid chromatography (HPLC).

In another general aspect, there is provided an amorphous form of daclatasvir dihydro chloride having purity of about 95% or more, of about 98% or more, of about 99% or more, of about 99.5% or more, of about 99.8% or more, of about 99.9% or more, as determined using high performance liquid chromatography (HPLC).

In general, the amorphous form of daclatasvir dihydrochloride having purity of about 99% or more is substantially free from one or more impurities, as determined using high performance liquid chromatography (HPLC). In general, the term “substantially free from impurities” includes total impurities of about 0.5% or less and single individual impurity of about 0.15% or less, determined using high performance liquid chromatography (HPLC).

In another general aspect, there is provided a process for the preparation of amorphous form of daclatasvir dihydrochloride, the process comprising:
(a) suspending daclatasvir dihydrochloride in one or more solvents:
(b) removing the solvent; and
(c) obtaining the amorphous form of daclatasvir dihydrochloride.

In general, the suspension of daclatasvir dihydrochloride may be a clear solution with homogenous mixture or a suspension or slurry with a heterogeneous mixture in one or more solvents selected from acetone, methanol, ethanol, ethyl acetate, butyl acetate, isopropyl acetate, acetonitrile, methylene dichloride, water-methanol or water-ethanol, water-acetone, dimethyl formamide, dimethylacetamide, dimethylsulfoxide and N-methylpyrrolidone.

In general, the weighed quantity of daclatasvir dihydrochloride is dissolved in 2-10 volumes of chosen solvent, particularly 4-5 volumes solvent at 25° C. to 30° C. The content may be stirred for 30 minutes at 25° C. to 30° C. The content may be filtered through Hyflosupercell, and filtrate is spray dried. The obtained powder may be further dried at 40° C. for 12-16 hours under vacuum to afford stable amorphous form of daclatasvir dihydrochloride.

In a preferred feature, the feedstock for spray drying is either a clear solution or in dispersion form.

In another general aspect, there is provided a pharmaceutical composition comprising an amorphous form of daclatasvir dihydrochloride substantially free residual solvents as measured by GC together with one or more pharmaceutically acceptable carriers, excipients or diluents.

In another general aspect, the amorphous form of daclatasvir dihydrochloride is stabilized by placing the amorphous daclatasvir dihydrochloride under nitrogen atmosphere in a double polythene bag and sealing it to obtain primary packing; placing the primary packing inside a double polyethylene bag optionally containing oxygen busters and sealing it to obtain secondary packing; placing the secondary packing inside a triple laminated bag optionally containing oxygen busters and sealing it; and placing the sealed triple laminated bag inside a high density polyethylene (HDPE) container and storing in controlled environment at a temperature of about 25° C. to 40° C. at a relative humidity from 60% RH to about 75% RH, respectively.

In another general aspect, there is provided a pharmaceutical composition comprising an amorphous daclatasvir dihydrochloride having at least one polymer together one or more of pharmaceutically acceptable carriers, excipients and diluents.

In another general aspect, there is provided a pharmaceutical composition comprising an amorphous form of daclatasvir dihydrochloride having one or more pharmaceutically acceptable carriers, excipients or diluents.

In another general aspect, there is provided a pharmaceutical composition comprising a storage stable amorphous form of daclatasvir dihydrochloride having one or more pharmaceutically acceptable carriers, excipients or diluents.

In another general aspect, there is provided an amorphous daclatasvir dihydrochloride having particle size distributions wherein the 10th volume percentile particle size (D10) is about 50 μm or less, the 50th volume percentile particle size (D50) is about 200 μm or less, and the 90th volume percentile particle size (D90) is about 400 μm or less, or any combination thereof.

In another general aspect, daclatasvir dihydrochloride to be used as the starting material may be prepared by the known methods reported in the prior i.e. by using the process as per U.S. Pat. No. 8,329,159 B2 as a reference.

The invention also encompasses pharmaceutical compositions comprising daclatasvir dihydrochloride of the invention. As used herein, the term “pharmaceutical compositions” includes pharmaceutical formulations like tablets, pills, powders, liquids, suspensions, emulsions, granules, capsules, suppositories, or injection preparations. Pharmaceutical compositions containing the daclatasvir dihydrochloride of the invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

Having described the invention with reference to certain preferred embodiments, other embodiments will become apparent to one skilled in the art from consideration of the specification.

### Example-1: Preparation of Amorphous Solid Dispersion of Daclatasvir Dihydrochloride of Formula (I) with Povidone (K-30 USP)

2.5 g (0.30 m mol) of daclatasvir dihydrochloride and 25 ml methanol were taken in round bottom flask at 25-30° C. 2.5 g of Povidone (K-30 USP) was added and stirred for 15 minutes. The reaction mixture was filtered and methanol was distilled under vacuum at 50° C. followed by co-distilled with n-heptane (2×25 ml). The residue was cooled to 25-30° C. and treated with n-Heptane (25 ml) and stirred for 30 minutes. The product was filtered and washed with n-Heptane (2×2.5 ml) and dried for 12 hours at 50-55° C. to obtain 4.4 g amorphous solid dispersion of daclatasvir dihydrochloride of Formula (I) with Povidone (K-30 USP). XRD as depicted in FIG. 1.

### Example 2: Preparation of Amorphous Form of Daclatasvir Dihydrochloride of Formula (I)

4 g (0.49 m mol) of daclatasvir dihydrochloride and 25 ml methanol were taken in round bottom flask at 25-30°. The reaction mass was stirred for 15 minutes. The reaction mixture was filtered and methanol was distilled under vacuum at 50° C. followed by co-distilled with n-heptane (2×4 ml). The residue was cooled to 25-30° C. and treated with n-Heptane (40 ml) and stirred for 30 minutes. The product was filtered and washed with n-Heptane (2×5 ml) and dried for 12 hours at 50-55° C. to obtain 3.4 g amorphous form of daclatasvir dihydrochloride of Formula (I). XRD as depicted in FIG. 2.

### Example 3: Preparation of Amorphous Form of Daclatasvir Dihydrochloride of Formula (I)

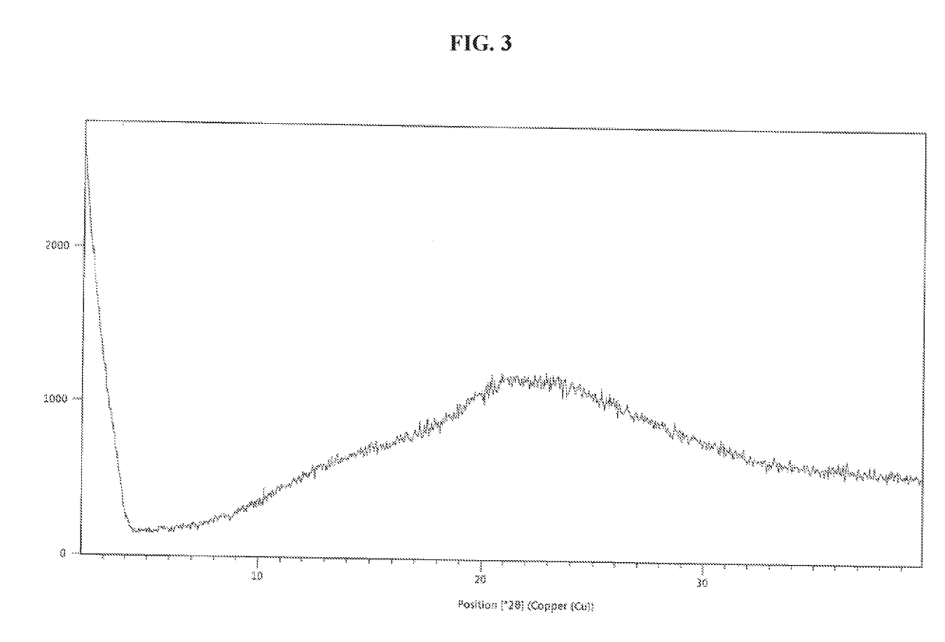

4 g (0.49 m mol) of daclatasvir dihydrochloride and 20 ml isopropyl alcohol were taken in round bottom flask at 25-30° C. The reaction mass was heated at 70-75° C. and 3.2 ml water was added and stirred for 15 minutes. The reaction mixture was cooled to 50° C. and solvent was distilled under vacuum at 50° C. The residue was treated with Heptane (40 ml) at 25-30° C. and stirred for 30 minutes. The product was filtered and washed with n-Heptane (2×5 ml) and dried for 12 hours at 50-55° C. to obtain 3.0 g amorphous form of daclatasvir dihydrochloride of Formula (I). XRD as depicted in FIG. 3.

### Example 4: Preparation of Amorphous Form of Daclatasvir Dihydrochloride of Formula (I)

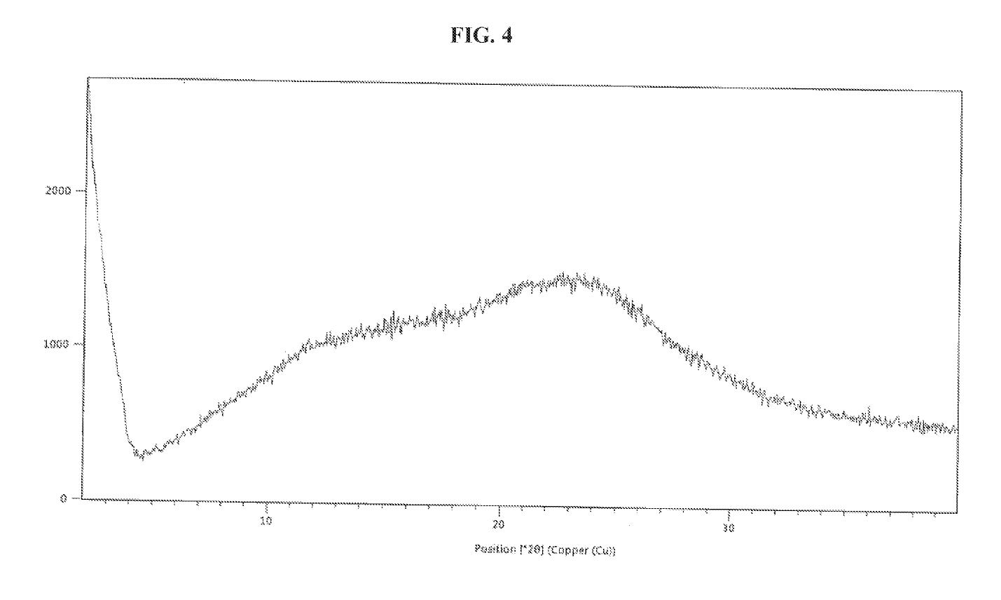

1 g (0.12 m mol) of daclatasvir dihydrochloride and 5 ml acetone were taken in round bottom flask at 25-30° C. The reaction mass was heated at 50-55° C. and stirred for 15 minutes. 1 ml water was added and stirred for 15 minutes. The reaction mixture was cooled to 25-35° C. and stirred for overnight. The solvent was distilled under vacuum at 50° C. and co-distilled by n-Heptane (2×2 ml). The residue was cooled to 25-30° C. and treated with Heptane (5 ml) and stirred for 30 minutes. The product was filtered and washed with n-Heptane (2×5 ml) and dried for 12 hours at 50-55° C. to obtain 0.7 g amorphous form of daclatasvir dihydrochloride of Formula (I). XRD as depicted in FIG. 4.

### Example 5: Preparation of Amorphous Form of Daclatasvir Dihydrochloride

13 g (0.16 m mol) of daclatasvir dihydrochloride and 130 ml methanol were dissolved at 25-30° C. to obtain a clear solution followed by spray drying in LU-222 Advanced model under below conditions.

Parameters of the spry dryer:

| Inlet temperature (° C.) | 75 | |
|---|---|---|
| Outlet temperature (° C.) | 70 | |
| Product temperature (° C.) | 65 | |
| Inlet high temperature (° C.) | 120 | |
| Outlet high temperature (° C.) | 100 | |
| Cooling temperature (° C.) | 50 | |
| Aspirator (Nm3/hr.) | 100 | |
| Feed pump (ml/min) | 3.0 | |
| D-block on time | 1 | Sec |
| D-block off time | 60 | sec |

| | |
|---|---|
| Cycle time (min.) | 999 |
| Air pressure (Kg/cm2) | 0.81 |

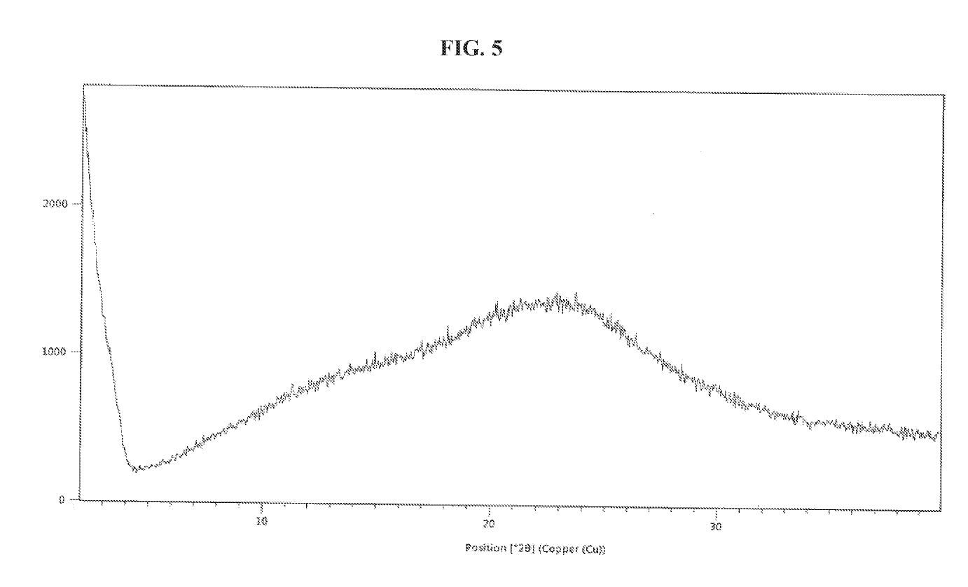

The product was collected from cyclone to obtain amorphous form of daclatasvir dihydrochloride of Formula (I). XRD as depicted in FIG. 5.

We claim:

1. An amorphous solid dispersion comprising daclatasvir dihydrochloride and a polymer.

2. The amorphous solid dispersion according to claim 1, wherein the polymer is selected from an ionic polymer or non-ionic polymer comprising hydroxypropylmethyl cellulose acetate succinate (HPMC-AS), hydroxypropylmethyl cellulose (HPMC), methacrylic acid copolymers, polyvinylpyrrolidone (PVP).

3. A process for the preparation of an amorphous solid dispersion according to claim 1, the process comprising mixing daclatasvir dihydrochloride with a polymer in one or more solvents and obtaining the amorphous solid dispersion daclatasvir dihydrochloride by removal of the solvent.

4. An amorphous form of daclatasvir dihydrochloride.

5. The amorphous form of daclatasvir dihydrochloride according to claim 4 having water content from about 0.5% to about 5% wt/wt.

6. An amorphous form of daclatasvir dihydrochloride substantially free from residual solvents.

7. A process for preparation an amorphous form of daclatasvir dihydrochloride according to claim 4, the process comprising:
(a) providing a solution of daclatasvir dihydrochloride in one or more solvents; and
(b) obtaining an amorphous form of daclatasvir dihydrochloride by removal of the solvent.

8. The process according to claim 8, wherein the solvent comprises one or more of alcohols selected from methanol, ethanol, isopropanol, 2-propanol, 1-butanol, and t-butyl alcohol; ketones selected from acetone, butanone, and methyl isobutyl ketone; esters selected from ethyl acetate, isopropyl acetate, t-butyl acetate, and isobutyl acetate; halogenated hydrocarbons selected from methylene dichloride, ethylene dichloride, and chlorobenzene; ethers selected from diethyl ether, dimethyl ether, diisopropyl ether and 1,4-dioxane; nitriles selected from acetonitrile, and propionitrile; and polar aprotic solvents selected from N,N-dimethylformamide, N,N-dimethylacetamide, N-methylpyrrolidone, dimethyl sulfoxide, and mixtures thereof.

9. The process according to claim 8, wherein the solvent is removed by one or more of rotational distillation device such as a Buchi Rotavapor, spray drying, agitated thin film drying (“ATFD”), and freeze drying (lyophilization).

10. The amorphous form of daclatasvir dihydrochloride according to claim 4 is stable that includes an amorphous form of daclatasvir dihydrochloride that doesn't convert to a crystalline form of daclatasvir dihydrochloride and free from residual solvents after the amorphous form of daclatasvir dihydrochloride is exposed to a relative humidity of 75% at 40° C. or 60% at 25° C. for a period of at least three months.

11. The stable amorphous form of daclatasvir dihydrochloride according to claim 10 is stabilized by placing the amorphous daclatasvir dihydrochloride under nitrogen atmosphere in a double polythene bag and sealing it to obtain primary packing; placing the primary packing inside a double polyethylene bag optionally containing oxygen busters and sealing it to obtain secondary packing; placing the secondary packing inside a triple laminated bag optionally containing oxygen busters and sealing it; and placing the sealed triple laminated bag inside a high density polyethylene (HDPE) container and storing in controlled environment at a temperature of about 25° C. to 40° C. at a relative humidity from 60% RH to about 75% RH, respectively.

12. The amorphous form of daclatasvir dihydrochloride according to claim 4, having purity of about 95% or more, of about 98% or more, of about 99% or more, of about 99.5% or more, of about 99.8% or more, of about 99.9% or more, as determined using high performance liquid chromatography (HPLC).

13. The amorphous form of daclatasvir dihydrochloride according to claim 12 is substantially free from one or more impurities, as determined using high performance liquid chromatography (HPLC).

14. The amorphous form of daclatasvir dihydrochloride according to claim 13 has total impurities of about 0.5% or less and single individual impurity of about 0.15% or less, determined using high performance liquid chromatography (HPLC).

15. The amorphous form of daclatasvir dihydrochloride according to claim 4, having particle size distributions wherein the 10th volume percentile particle size (D10) is about 50 μm or less, the 50th volume percentile particle size (D50) is about 200 μm or less, and the 90th volume percentile particle size (D90) is about 400 μm or less, or any combination thereof.

16. A process for the preparation of amorphous form of daclatasvir dihydrochloride according to claim 4, the process comprising:
(a) suspending daclatasvir dihydrochloride in one or more solvents;
(b) removing the solvent; and
(c) obtaining the amorphous form of daclatasvir dihydrochloride.

17. The process according to claim 16, wherein the solvent comprises one or more of acetone, methanol, ethanol, ethyl acetate, butyl acetate, isopropyl acetate, acetonitrile, methylene dichloride, water-methanol or water-ethanol, water-acetone, dimethyl formamide, dimethylacetamide, dimethylsulfoxide and N-methylpyrrolidone.

18. A pharmaceutical composition comprising an amorphous solid dispersion of daclatasvir dihydrochloride according to claim 1 having one or more pharmaceutically acceptable carriers, excipients or diluents.

19. A pharmaceutical composition comprising an amorphous daclatasvir dihydrochloride according to claim 4 having one or more of pharmaceutically acceptable carriers, excipients and diluents.

20. A pharmaceutical composition comprising an amorphous form of daclatasvir dihydrochloride according to claim 6 having one or more pharmaceutically acceptable carriers, excipients or diluents.
